(19) United States
(12) Patent Application Publication
(10) Pub. No.: US 2014/0178908 A1
(43) Pub. Date: Jun. 26, 2014

(54) IMMUNOASSAY AND IMMUNOASSAY APPARATUS

(71) Applicant: SYSMEX CORPORATION, KOBE-SHI (JP)

(72) Inventors: Keiko YOSHIKAWA, Kobe-shi (JP); Naoto KOSHIMURA, Kobe-shi (JP)

(73) Assignee: SYSMEX CORPORATION, KOBE-SHI (JP)

(21) Appl. No.: 14/109,442

(22) Filed: Dec. 17, 2013

(30) Foreign Application Priority Data
Dec. 21, 2012 (JP) 2012-279949

(51) Int. Cl. G01N 33/543

(52) U.S. Cl. CPC G01N 33/543
USPC 435/7.93; 436/501; 435/287.2; 422/69

(57) ABSTRACT

A method of competitive immunoassay is provided. The method comprising: creating a first calibration curve created for a first reaction time period and a second calibration curve created for a second reaction time period longer than the first reaction time period; preparing a measurement sample containing a target substance in a specimen, a reactant, and a competitive substance; obtaining a reference information related to the concentration of the target substance after the first reaction time period since the preparation of the measurement sample; first outputting the concentration obtained based on the first calibration curve as a measurement result in the case where the reference information shows that the concentration of the target substance is less than a predetermined threshold; and second outputting the concentration obtained based on the second calibration curve as the measurement result in the case where the reference information obtained in the obtaining is information showing that the concentration of the target substance is more than or equal to the threshold.

### BACKGROUND OF THE INVENTION

### Field of the Invention

The present invention relates to a competitive immunoassay and an immunoassay apparatus.

### BACKGROUND

Conventionally, the competitive immunoassay has been known as a procedure for measuring the concentration of small amounts of antigens and antibodies contained in a sample.

For example, a procedure described in Patent Document 1 is known as a procedure for extending a measurable concentration range in the competitive immunoassay and the immunoassay apparatus. In the procedure described in Japanese Patent Application Laid-Open (JP-A) No. 2011-80975, a plurality of calibration curves of different concentrations of a molecular recognition reagent are previously created, the calibration curve to be used for concentration conversion is selected depending on the absorbance of a measurement target specimen. Thus, in the procedure described in Patent Document 1, a measurable concentration range is extended by previously creating the plurality of calibration curves of different concentration ranges.

However, in the procedure of Patent Document 1, it is necessary to create the calibration curves of mutually different concentrations of the molecular recognition reagent. Accordingly, there are problems such that the usage of a calibrator necessary for creating calibration curves and the amount of the reagent are increased, and further the user's work rate necessary for creating calibration curves is increased. Furthermore, it is necessary to design a measurement device corresponding to the step of preparing a sample having a plurality of concentrations. Therefore, a problem such that the configuration of the measurement device is complicated is caused.

### SUMMARY OF THE INVENTION

The scope of the present invention is defined solely by the appended claims, and is not affected to any degree by the statements within this summary.

A first aspect of the present invention is a method of competitive immunoassay. The method comprises: creating a first calibration curve created for a first reaction time period and a second calibration curve created for a second reaction time period longer than the first reaction time period; preparing a measurement sample containing a target substance in a specimen, a reactant, and a competitive substance; obtaining a reference information related to the concentration of the target substance after the first reaction time period since the preparation of the measurement sample; first outputting the concentration obtained based on the first calibration curve as a measurement result in the case where the reference information shows that the concentration of the target substance is less than a predetermined threshold; and second outputting the concentration obtained based on the second calibration curve as the measurement result in the case where the reference information obtained in the obtaining is information showing that the concentration of the target substance is more than or equal to the threshold.

A second aspect of the present invention is an apparatus for competitive immunoassay. The apparatus comprises: a sample preparation unit for preparing a measurement sample containing a target substance in a specimen, a reactant, and a competitive substance; a detection unit; and a control unit. The control unit: stores a first calibration curve created for a first reaction time period and a second calibration curve created for a second reaction time period longer than the first reaction time period; obtains predetermined reference information related to the concentration of the target substance after the first reaction time period since the preparation of the measurement sample based on the output of the detection unit; outputs the concentration obtained based on the first calibration curve as the measurement result in the case where the obtained reference information shows that the concentration of the target substance is less than a predetermined threshold; and outputs the concentration obtained based on the second calibration curve as the measurement result in the case where the reference information shows that the concentration of the target substance is more than or equal to the threshold.

A third aspect of the present invention is an apparatus for competitive immunoassay. The apparatus comprises: a sample preparation unit configured to prepare a measurement sample containing a target substance in a specimen, a reactant, and a competitive substance; a detection unit; and a control unit. The control unit: stores a first calibration curve created for a first reaction time period and a second calibration curve created for a second reaction time period longer than the first reaction time period; obtains the concentration of the target substance after the first reaction time period since the preparation of the measurement sample based on the output of the detection unit; outputs the concentration obtained based on the first calibration curve as the measurement result in the case where the obtained concentration shows that the concentration of a target substance is less than a predetermined threshold; and outputs the concentration obtained based on the second calibration curve as the measurement result in the case where the obtained concentration shows that the concentration of the target substance is more than or equal to the threshold.

### BRIEF DESCRIPTION OF THE DRAWINGS

FIGS. 1A and 1B show external and internal configurations of a biological sample analyzer according to an embodiment;

FIGS. 2A to 2C schematically show configurations of a sample preparation unit, a measurement unit, and a sheath flow cell according to the embodiment;

FIG. 3 shows the configuration of the biological sample analyzer according to the embodiment;

FIGS. 4A to 4D show a procedure for obtaining the concentration of a target substance according to the embodiment;

FIGS. 5A to 5C are a flow chart showing a step of creating calibration curves according to the embodiment, a diagram for describing the degree of aggregation, and a diagram for describing the calibration curves;

FIG. 6 is a flow chart showing a step of measuring a specimen according to the embodiment;

FIGS. 7A to 7C are diagrams for describing the concentration to be obtained according to the embodiment;

FIGS. 8A and 8B are flow charts showing a step of setting according to the embodiment;

FIGS. 9A to 9C are diagrams for describing the results in which, when a plurality of calibration curves according to the embodiment are created, ranges of the aggregation degree suitable for using the calibration curves (concentration ranges) are examined;

FIG. 10 is a flow chart showing a step of measuring a specimen according to a modification; and

FIGS. 11A and 11B are flow charts showing the step of measuring a specimen according to another modification.

### DETAILED DESCRIPTION OF PREFERRED EMBODIMENTS

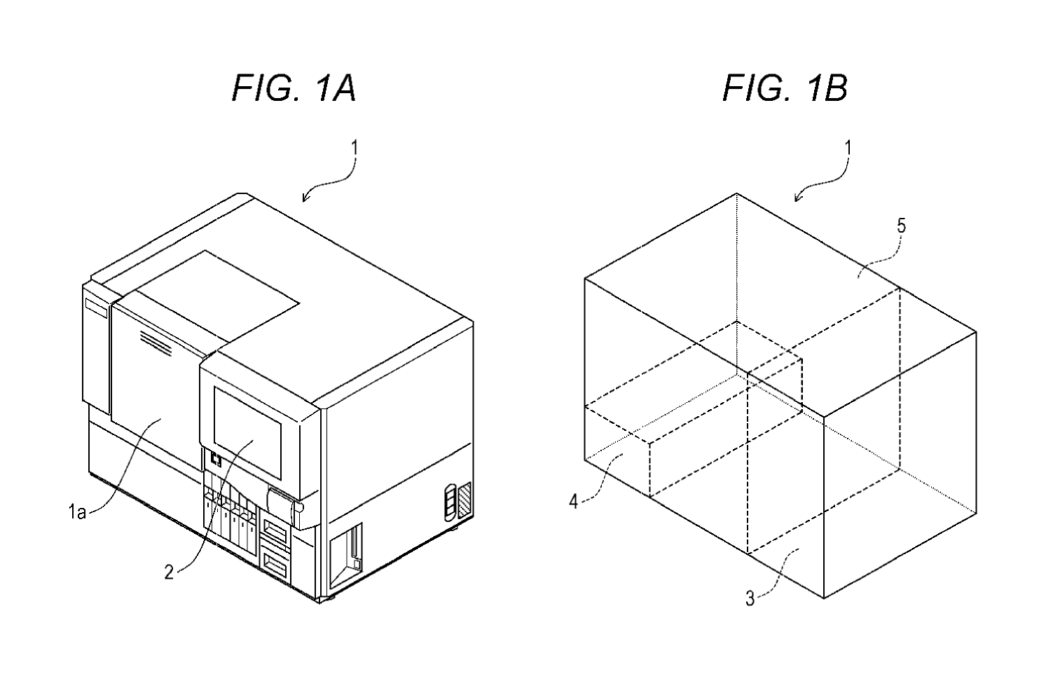

FIG. 1A is a view showing the external configuration of a biological sample analyzer 1, and FIG. 1B is a view showing the internal configuration of the biological sample analyzer 1.

A cover 1a and a display input unit 2 composed of a touch panel are disposed on the front surface of the biological sample analyzer 1. A control unit 3 for controlling each unit is disposed in a space at the right side in the biological sample analyzer 1, and a measurement unit 4 for detecting a signal from a measurement sample is disposed in a space at the lower left side in the biological sample analyzer 1. Further, a sample preparation unit 5 for preparing a measurement sample is disposed in the remaining space in the biological sample analyzer 1.

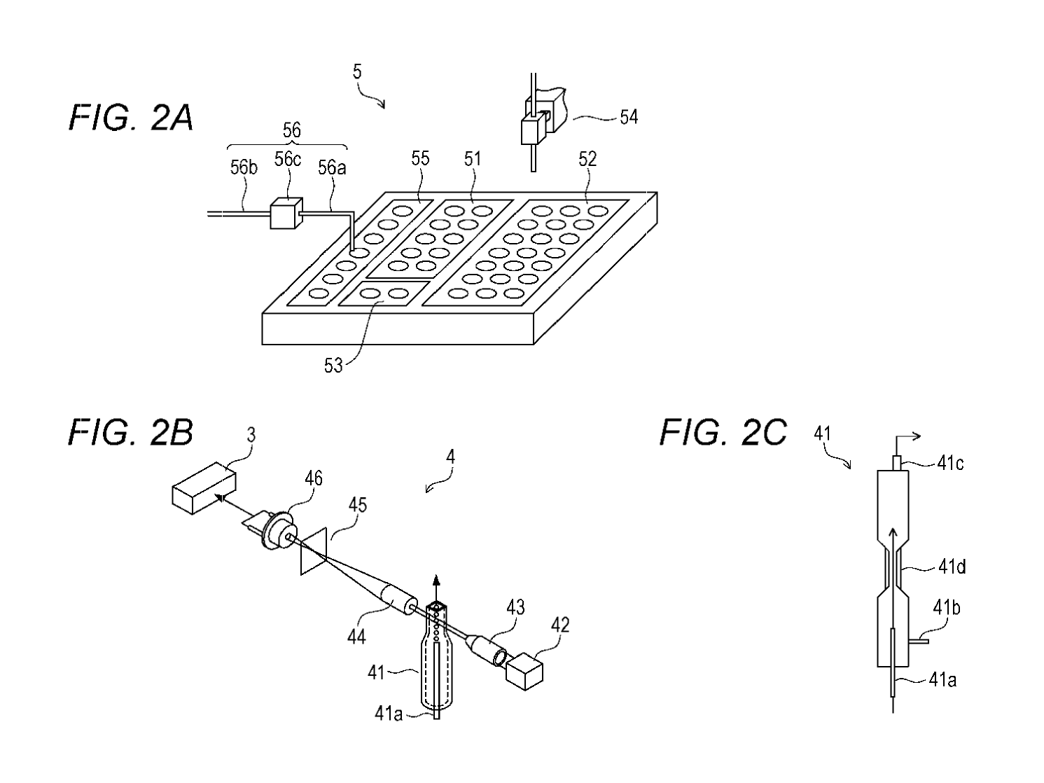

FIG. 2A is a view schematically showing the configuration of the sample preparation unit 5.

The sample preparation unit 5 includes a specimen setting unit 51, a standard sample setting unit 52, a reagent setting unit 53, a dispensing device 54, a reaction unit 55, and a liquid feeding device 56. A user opens the cover 1a to set various containers to the specimen setting unit 51, the standard sample setting unit 52, and the reagent setting unit 53.

A container for housing a specimen containing a target substance is set in the specimen setting unit 51. A container for housing a standard sample (calibrator) which contains a target substance and has a known concentration is set in the standard sample setting unit 52. In this embodiment, ten standard samples (1) to (10) having different concentrations of the target substance are used. 10 containers for housing the standard samples are set in the standard sample setting unit 52. A container for housing a reaction buffer solution containing a reactant and a container for housing a reagent containing a competitive substance (hereinafter, referred to as “competitive reagent”) are set in the reagent setting unit 53. An empty cuvette is set in the reaction unit 55.

In this embodiment, the target substance is an antigen. Specifically, it is thyroxine (T4) which is a type of thyroid hormone. The standard samples (1) to (10) are prepared by adding thyroxine (manufactured by Nacalai Tesque, Inc.) to normal human serum from which triiodothyronine (T3) and thyroxine (T4) are removed (manufactured by CC Biotech Corporation). The concentration of thyroxine in the standard samples (1) to (10) is set as shown in Table 1 below.

TABLE 1

| Standard sample | Thyroxine concentration (μg/dL) |
|---|---|
| (1) | 0 |
| (2) | 0.25 |
| (3) | 0.5 |
| (4) | 1 |
| (5) | 2.5 |
| (6) | 5 |
| (7) | 10 |
| (8) | 25 |
| (9) | 50 |
| (10) | 100 |

Further, the reactant is an antibody. Specifically, it is an anti-thyroxine antibody. The reaction buffer solution is prepared by adjusting the pH of 16 mg/mL of 3,3-dimethyl glutaric acid, 1 mg/mL of 2-amino-2-methyl-1,3-propanediol, 12 mg/mL of tris, 1.5% (w/v) BSA, 1.5% (w/v) PVA105 (manufactured by Kuraray Co., Ltd.), 0.58% (w/v) NaCl, 0.016% (w/v) NaN₃, 0.2 mg/mL of 8-anilino-1-naphthalenesulfonic acid ammonium, and 0.5 μg/mL of anti-thyroxine antibody (manufactured by Medix Biochemica) to 7.1.

Further, the competitive substance is a labeled antigen. Specifically, it is a triiodothyronine-sensitized latex produced by sensitizing triiodothyronine (T3) to polystyrene latex particles. The competitive reagent is prepared by sensitizing triiodothyronine (BSA-X-T3; manufactured by Michigan Diagnostics) to polystyrene latex particles having a diameter of 0.8 μm to produce a triiodothyronine-sensitized latex, adding 25 mM of an MOPSO buffer (MOPSO (manufactured by Dojindo Laboratories), 30 mM of NaCl, 0.1% (w/v) NaN₃, 6% (w/v) sucrose, and 2% (w/v) BSA, pH 7.1) to the produced triiodothyronine-sensitized latex so as to be 1% (w/v).

Subsequently, the dispensing device 54 aspirates and discharges a predetermined amount of liquid from the tip thereof and is configured to be able to move in side-to-side, up-and-down, and back-and-forth directions by a driving device (not shown). The dispensing device 54 appropriately dispenses a specimen, a standard sample, a reaction buffer solution, and a competitive reagent into the cuvette of the reaction unit 55.

The reaction unit 55 includes a temperature regulating mechanism (not shown) for maintaining a constant temperature of the solution in the cuvette and a stirring mechanism (not shown) for stirring the solution in the cuvette. The specimen, the reaction buffer solution, and the competitive reagent are mixed in the cuvette set in the reaction unit 55 to prepare a measurement sample. Before the specimen measurement, the standard sample, the reaction buffer solution, and the competitive reagent are mixed in the cuvette set in the reaction unit 55 to prepare a measurement sample for creating calibration curves.

If the reaction buffer solution containing the reactant (antibody) is mixed with the competitive reagent containing the competitive substance (labeled antigen), the reactant and the competitive substance are aggregated by the antigen-antibody reaction. If the specimen or the standard sample, the reaction buffer solution and the competitive reagent are mixed, the target substance (antigen) in the specimen or the standard sample and the competitive substance (labeled antigen) in the competitive reagent are competitively reacted with the reactant (antibody) in the reaction buffer solution. That is, the biological sample analyzer 1 allows the antigen-antibody reaction to be competitively caused by the competitive assay based on the latex agglutination. The measurement unit 4 detects aggregates composed of the reactant and the competitive substance.

The liquid feeding device 56 is composed of an aspiration tube 56a for aspirating a measurement sample, a liquid feeding tube 56b which feeds the measurement sample aspirated from the aspiration tube 56a to the measurement unit 4, and a pump 56c which aspirates the measurement sample and feeds it to the measurement unit 4. The liquid feeding device 56 is configured to be able to move in side-to-side, up-and-down, and back-and-forth directions by a driving device (not shown). A predetermined amount of the measurement sample in the cuvette set in the reaction unit 55 is fed to the measurement unit 4 by the liquid feeding device 56.

FIG. 2B is a view schematically showing the configuration of the measurement unit 4, and FIG. 2C is a view schematically showing the configuration of the sheath flow cell 41.

The measurement unit 4 includes a flow cell 41, a laser light source 42, a condenser lens 43, a light collecting lens 44, a pinhole 45, and a photo diode 46. The flow cell 41 passes the measurement sample prepared by the sample preparation unit 5 in a state of being enclosed in a sheath liquid, and as shown in FIG. 2C, includes a sample nozzle 41a which injects the measurement sample upward toward a pore portion 41d, a sheath liquid supply port 41b, and a drainage port 41c.

Laser light emitted from the laser light source 42 passes through the condenser lens 43 and is delivered to the pore portion 41d of the flow cell 41. Thus, the measurement sample passing through the inside of the pore portion 41d is irradiated with the laser light. The light collecting lens 44 focuses forward scattered light from each of the particles in the measurement sample irradiated with the laser light. The photo diode 46 receives the forward scattered light passed through the pinhole 45, performs photoelectric conversion of the received forward scattered light, and produces a forward scattered light signal. The produced forward scattered light signal is sent to the control unit 3.

Here, if aggregates (agglomerated particles) produced by the antigen-antibody reaction of the reactant (antibody) and the competitive substance (labeled antigen) are compared with other particles not aggregated (single particles), the agglomerated particles are larger than the single particles. Thus, the control unit 3 can determine whether particles passed through the pore portion 41d of the flow cell 41 are agglomerated or single particles based on the magnitude of the forward scattered light signal. The control unit 3 can separately calculate single particles and agglomerated particles based on the received forward scattered light signal so that the aggregation degree can be determined. As the aggregation degree in this embodiment, a value of P/T calculated based on the number of single particles (M), the number of agglomerated particles (P), and the total particle number (T) (the total of M and P) is used.

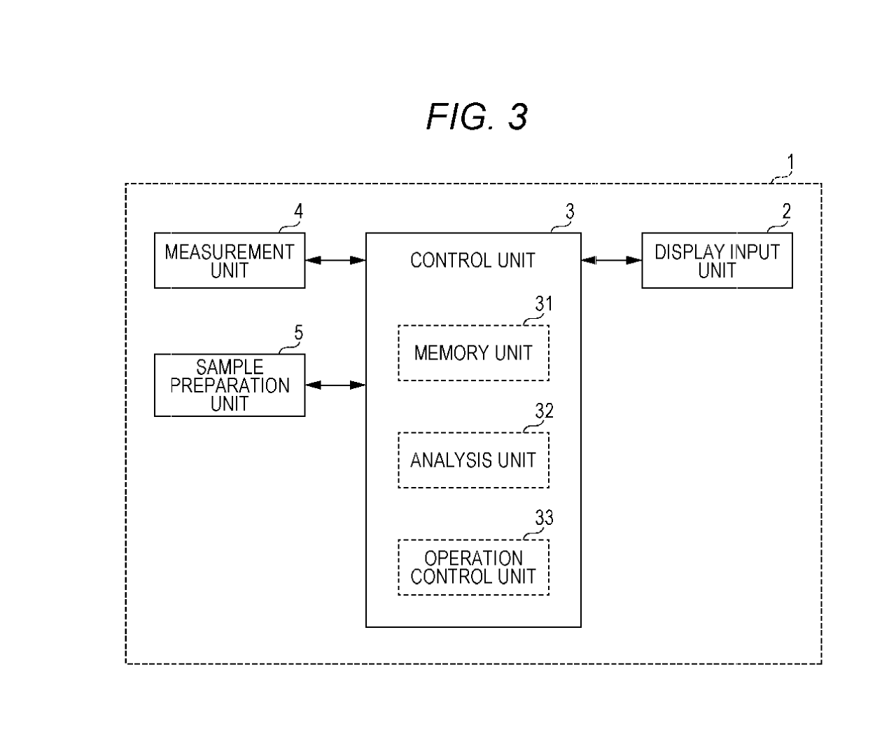

FIG. 3 is a view showing the configuration of the biological sample analyzer 1.

The control unit 3 includes a microcomputer having a storage device such as CPU, ROM or RAM and a circuit which processes various signals. Thus, the control unit 3 has functions of a memory unit 31, an analysis unit 32, and an operation control unit 33.

The memory unit 31 stores an analysis program which analyzes the forward scattered light signal and a control program which controls the operation of each of the devices. The memory unit 31 stores data of the received forward scattered light signal, the step results by the analysis program, data of calibration curves, and various set contents. The analysis unit 32 analyzes the forward scattered light signal based on the analysis program and calculates the concentration of the target substance contained in the measurement sample. The calculation results by the analysis unit 32 are output to the display input unit 2. The operation control unit 33 controls the operation of the units of the devices based on the control program stored in the memory unit 31.

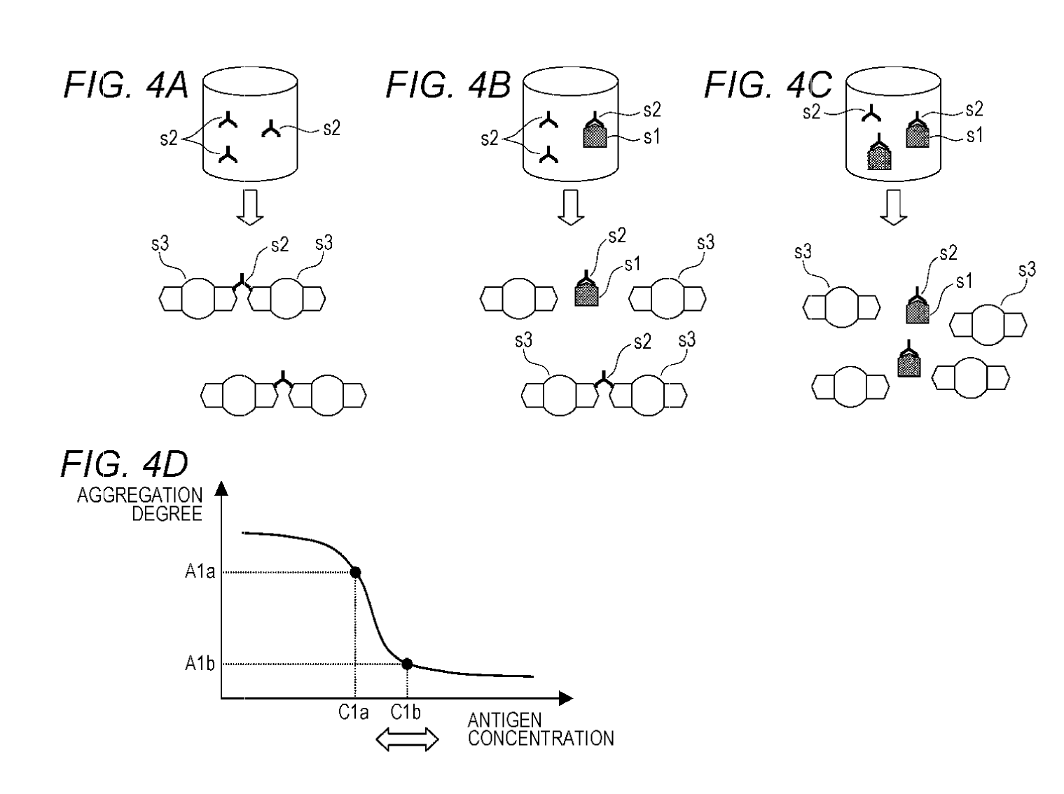

FIGS. 4A to 4D are views for describing the procedure for obtaining the concentration of a target substance. Here, the antigen (target substance) is represented by s1, the antibody (reactant) in the reaction buffer solution is represented by s2, and the competitive substance (labeled antigen) in the competitive reagent is represented by s3. FIGS. 4A to 4C schematically show antigen-antibody reactions in the case where the concentration of the antigen s1 is low, medium or high. The upper row of FIGS. 4A to 4C shows states in which the reaction buffer solution is mixed with a sample containing the antigen s1 in an empty cuvette. The lower row of FIGS. 4A to 4C shows states in which a mixture of the sample and the reaction buffer solution in the cuvette is mixed with the competitive reagent.

With reference to FIG. 4A, in the case where the concentration of the antigen s1 is low, even if the sample and the reaction buffer solution are mixed, many antibodies s2 are present without binding to the antigen s1 in the sample as shown in the upper row. If the competitive reagent is added to the cuvette in this state and a predetermined time is passed, lots of the antibodies s2 and the competitive substances s3 are aggregated as shown in the lower row.

With reference to FIG. 4B, in the case where the concentration of the antigen s1 is medium, if the sample and the reaction buffer solution are mixed, a part of the antibodies s2 is bound to the antigen s1 in the sample as shown in the upper row. If the competitive reagent is added to the cuvette in this state and a predetermined time is passed, a part of the antibodies s2 and the competitive substances s3 are aggregated as shown in the lower row.

With reference to FIG. 4C, in the case where the concentration of the antigen s1 is high, if the sample and the reaction buffer solution are mixed, lots of the antibodies s2 are bound to the antigens s1 in the sample as shown in the upper row. If the competitive reagent is added to the cuvette in this state and a predetermined time is passed, the antibodies s2 and the competitive substances s3 are hardly aggregated as shown in the lower row.

Thus, the aggregation degree of the antibody s2 and the competitive substance s3 changes depending on the concentration of the antigen s1 in the sample. The aggregation of the antibody s2 and the competitive substance s3 progresses according to the reaction time.

In the specimen measurement, a calibration curve is first created using the standard samples (1) to (10). Here, the reaction buffer solution is mixed with each of the standard samples having a predetermined concentration. Further, the competitive reagent is mixed. The sample thus prepared is measured by the measurement unit 4 at the timing after a predetermined reaction time.

As described with reference to FIGS. 2B and 2C, the measurement unit 4 performs photoelectric conversion of the forward scattered light produced by irradiation with laser light and outputs the produced forward scattered light signal to the control unit 3. The control unit 3 plots the aggregation degree calculated based on the received forward scattered light signal and the known concentration (concentration of the antigen s1) of the standard sample used in this case on a graph. Thus, the control unit 3 repeatedly performs the operation on the standard samples (1) to (10) and creates a calibration curve corresponding to a predetermined reaction time as shown in FIG. 4D.

In the specimen measurement, the reaction buffer solution is mixed with the specimen, and further the competitive reagent is mixed. When the predetermined reaction time is passed, the concentration of the antigen s1 contained in the specimen is obtained on the basis of the aggregation degree calculated based on the forward scattered light from the specimen and the previously created calibration curve corresponding to the predetermined reaction time. For example, in FIG. 4D, in the case where the aggregation degree calculated in the specimen measurement is A1a, the concentration of the antigen s1 is C1a. In the case where the aggregation degree calculated in the specimen measurement is A1b, the concentration of the antigen s1 is C1b.

However, the slope (gradient) of the created calibration curve is generally large near the center, but it is small near both ends as shown in FIG. 4D. Accordingly, in the case where the aggregation degree obtained in the specimen measurement is A1b, the concentration C1b largely varies depending on slight variations in the aggregation degree A1b. Thus, the accuracy of the concentration C1b to be obtained becomes low. In this embodiment, the biological sample analyzer 1 is configured such that two calibration curves corresponding to different reaction times are previously created before the specimen measurement, and a suitable calibration curve is used properly depending on the aggregation degree calculated in the specimen measurement.

Hereinafter, a procedure for creating two calibration curves, using the calibration curves properly, and obtaining the concentration of the target substance with high accuracy will be described.

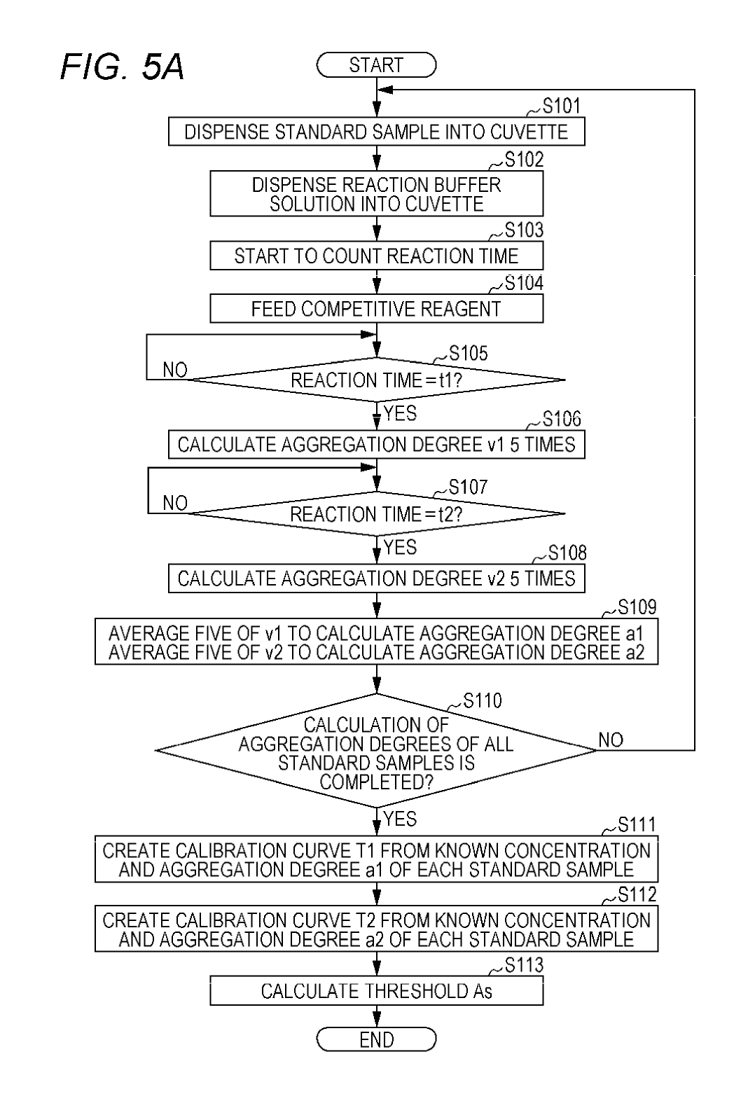

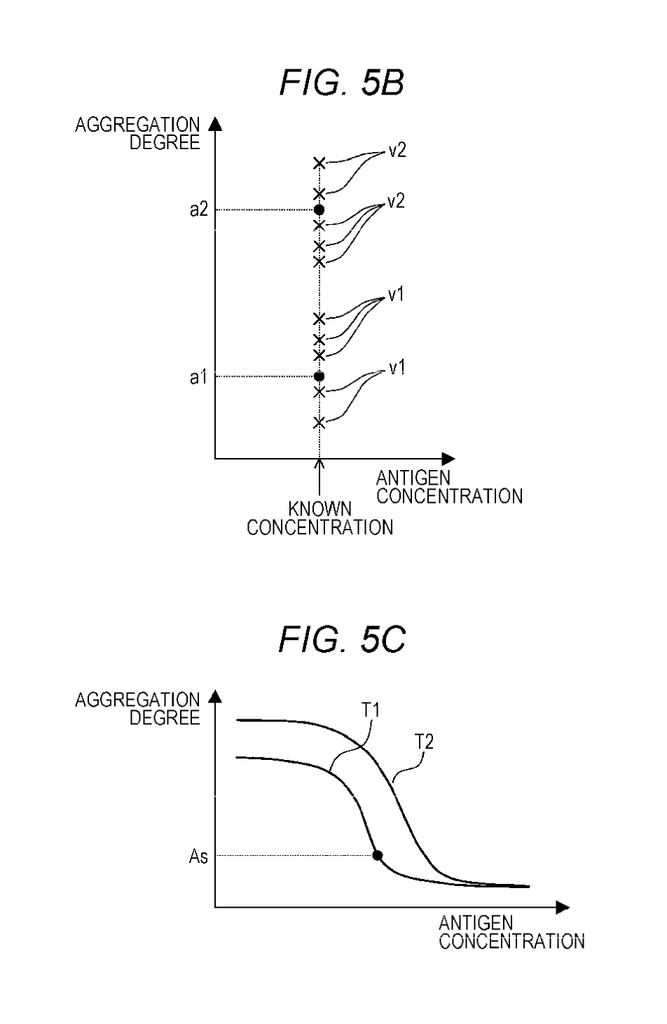

FIG. 5A is a flow chart showing the step of creating calibration curves by the control unit 3.

The control unit 3 first dispenses a predetermined amount (10 μL in this embodiment) of one of the standard samples (1) to (10) into an empty cuvette set in the reaction unit 55 (S101). Subsequently, the control unit 3 dispenses a predetermined amount (80 μL in this embodiment) of the reaction buffer solution into the cuvette (S102), and starts to count the reaction time after dispensing the reaction buffer solution (S103). Subsequently, the control unit 3 feeds a predetermined amount (10 μL in this embodiment) of the competitive reagent to the cuvette when the reaction time reaches a predetermined time (50 seconds in this embodiment) (S104). Then, the control unit 3 heats the cuvette to a predetermined temperature (45° C. in this embodiment) by the reaction unit 55 and waits the step until the reaction time reaches t1 (320 seconds in this embodiment) (S105).

When the reaction time reaches t1 (S105: YES), the control unit 3 feeds only a predetermined amount of the measurement sample in the cuvette to the measurement unit 4, executes the measurement by the measurement unit 4, and calculates an aggregation degree v1 of the measurement sample 5 times (S106). Subsequently, the control unit 3 waits the step until the reaction time reaches t2 (1370 seconds in this embodiment) (S107). When the reaction time reaches t2 (S107: YES), the control unit 3 feeds only a predetermined amount of the measurement sample in the cuvette to the measurement unit 4, executes the measurement by the measurement unit 4, and calculates an aggregation degree v2 of the measurement sample 5 times (S108).

Subsequently, the control unit 3 averages the five aggregation degrees v1 of the standard sample to calculate an aggregation degree a1 as shown in FIG. 5B. Similarly, the control unit 3 averages the five aggregation degrees v2 of the standard sample to calculate an aggregation degree a2 as shown in FIG. 5B (S109). Then, the control unit 3 performs steps S101 to S109 until the aggregation degrees a1 and a2 of all the standard samples are calculated (S110).

When the calculation of the aggregation degrees of all the standard samples is completed (S110: YES), the control unit 3 creates a calibration curve T1 as shown in FIG. 5C from the known concentrations of the standard samples (1) to (10) and the calculated aggregation degrees a1 of the standard samples (1) to (10) (S111). Further, the control unit 3 creates a calibration curve T2 as shown in FIG. 5C from the known concentrations of the standard samples (1) to (10) and the calculated aggregation degrees a2 of the standard samples (1) to (10) (S112). Since the calibration curve T2 is created based on the aggregation degrees a2 of the standard samples when the reaction time is t2 larger than t1, it is created above the calibration curve T1 as shown in FIG. 5C.

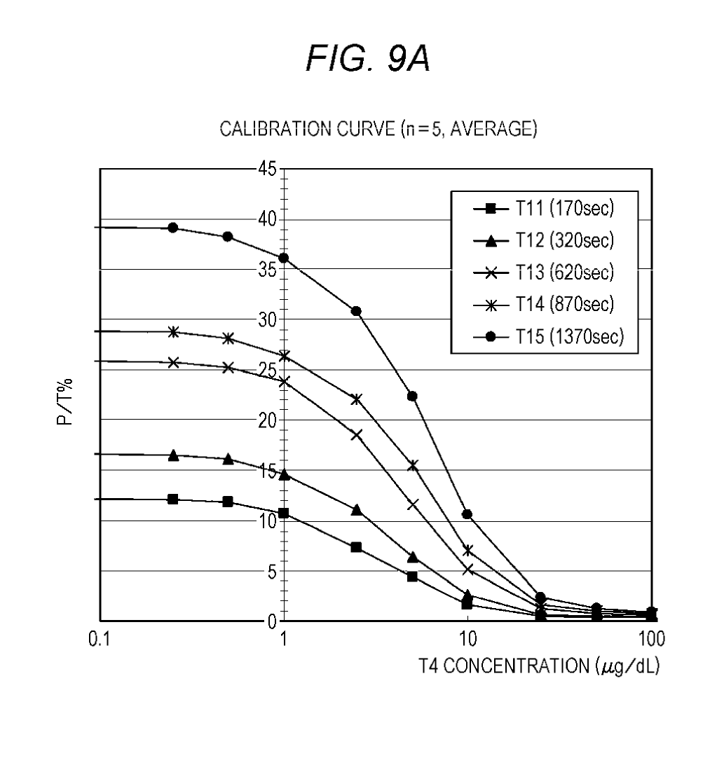

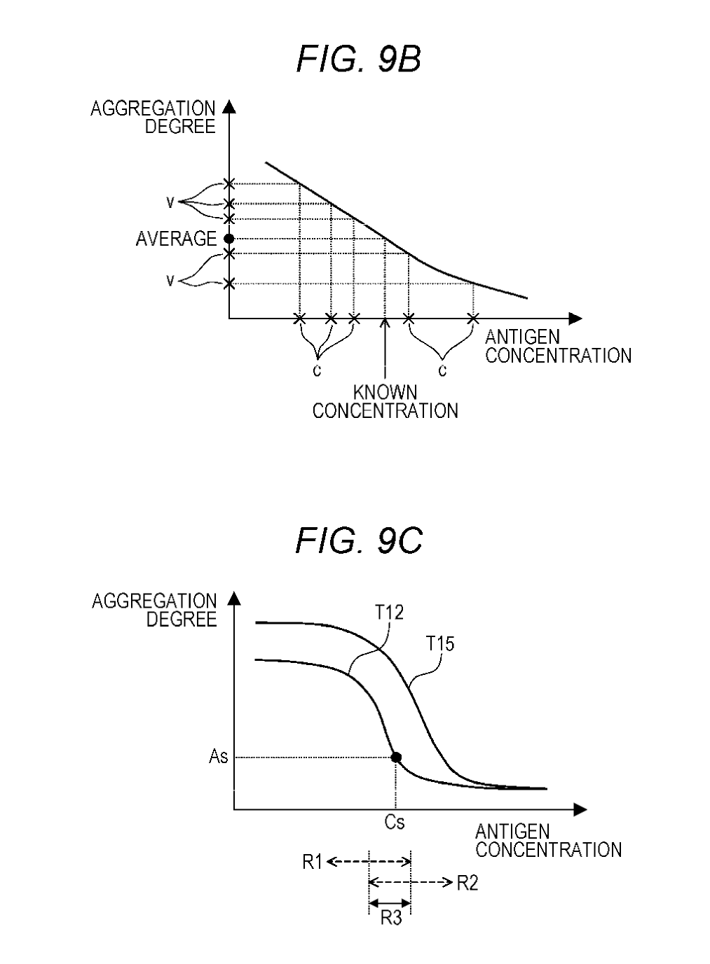

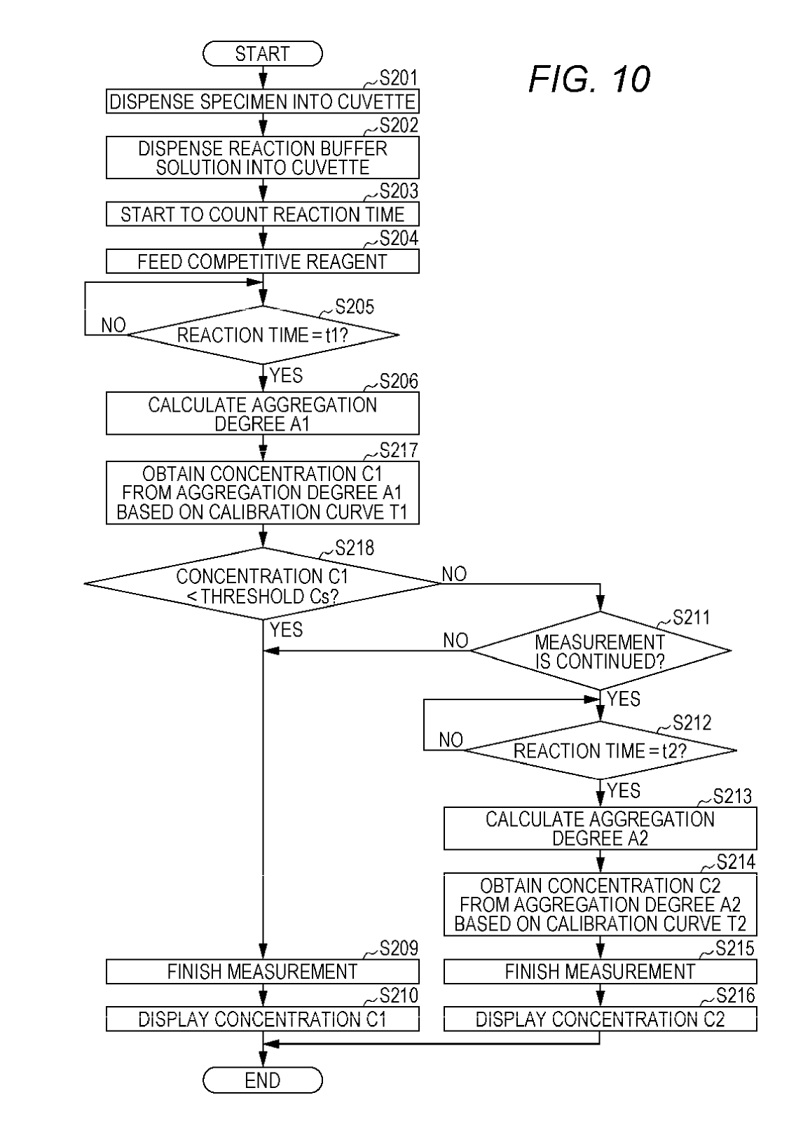

Subsequently, the control unit 3 calculates a threshold As related to the aggregation degree for determining which the calibration curves T1 and T2 are used in order to obtain the concentration of a specimen in the step of measuring a specimen to be described below (S113). The procedure for calculating the threshold As will be described later with reference to FIGS. 9A to 9C. In this manner, the step of creating calibration curves is terminated.

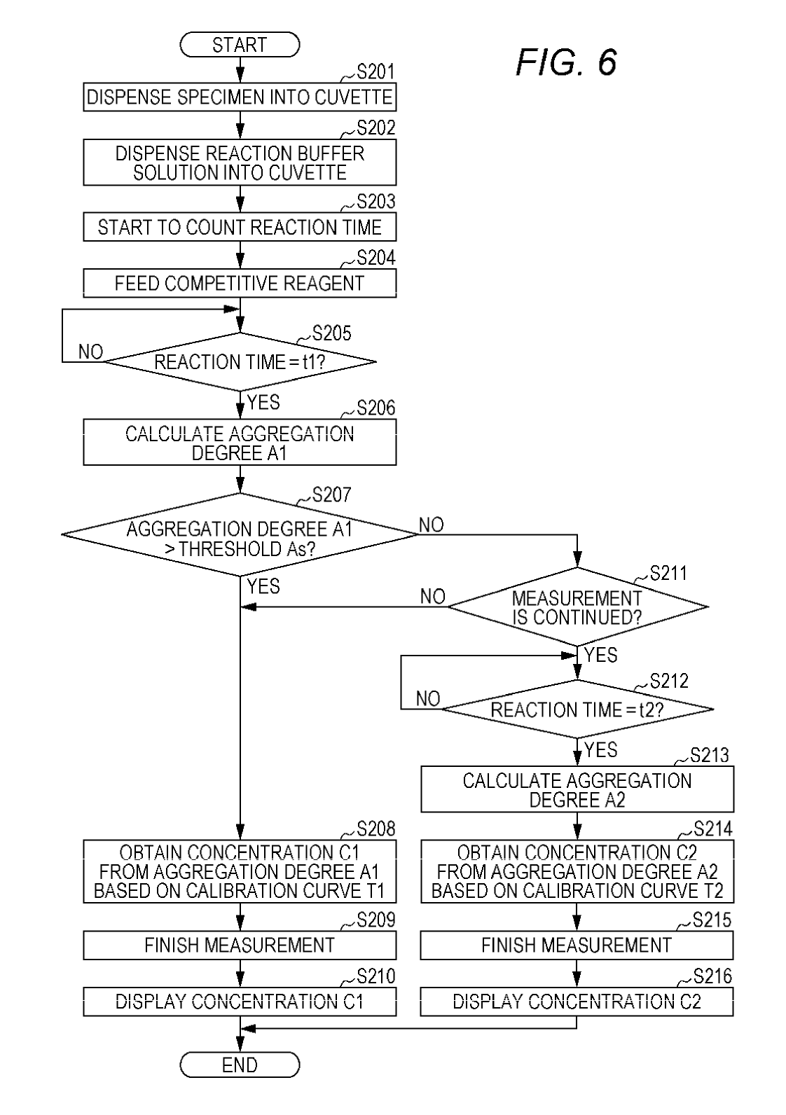

FIG. 6 is a flow chart showing the step of measuring a specimen by the control unit 3.

The control unit 3 first dispenses a predetermined amount (10 μL in this embodiment) of the specimen into an empty cuvette set in the reaction unit 55 (S201). Subsequently, the control unit 3 dispenses a predetermined amount (80 μL in this embodiment) of the reaction buffer solution into the cuvette (S202), and starts to count the reaction time after dispensing the reaction buffer solution (S203). Subsequently, the control unit 3 feeds a predetermined amount (10 μL in this embodiment) of the competitive reagent to the cuvette when the reaction time reaches a predetermined time (50 seconds in this embodiment) (S204). Then, the control unit 3 heats the cuvette to a predetermined temperature (45° C. in this embodiment) by the reaction unit 55 and waits the step until the reaction time reaches t1 (S205). When the reaction time reaches t1 (S205: YES), the control unit 3 feeds only a predetermined amount of the measurement sample in the cuvette to the measurement unit 4, executes the measurement by the measurement unit 4, and calculates an aggregation degree A1 of the measurement sample (S206).

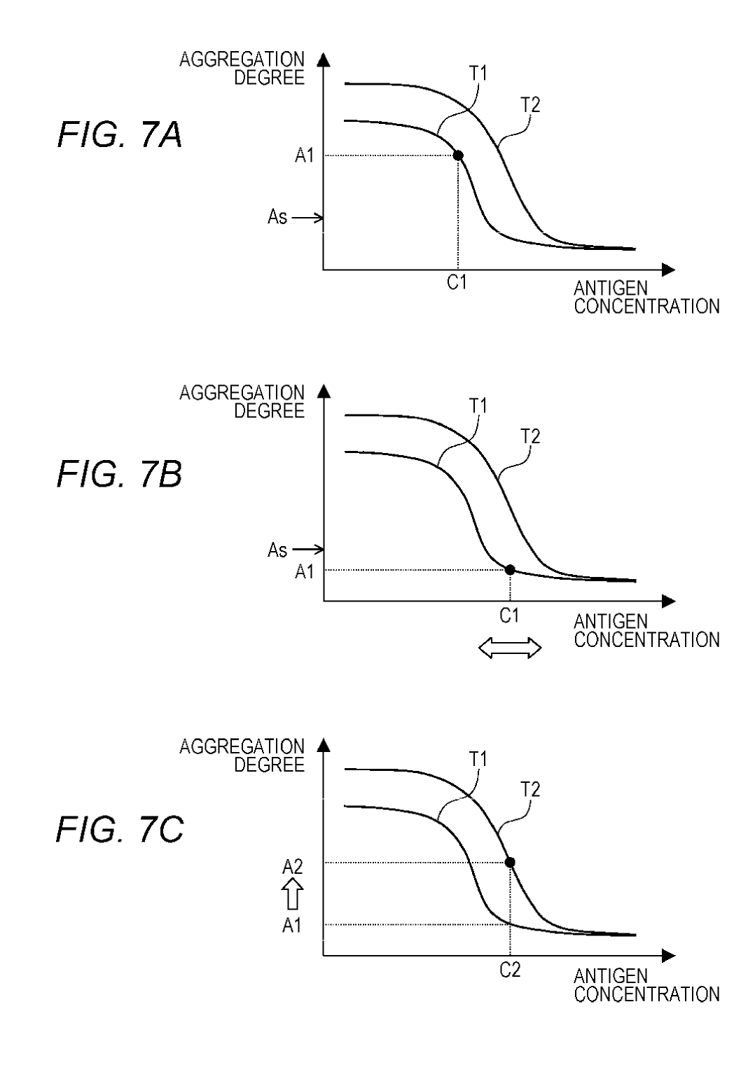

Subsequently, the control unit 3 determines whether the calibration curve T1 created previously is used (S207). Specifically, as shown in FIG. 7A, it is determined to use the calibration curve T1 in the case where the aggregation degree A1 is larger than the threshold As. On the other hand, as shown in FIG. 7B, it is determined not to use the calibration curve T1 in the case where the aggregation degree A1 is less than or equal to the threshold As.

When the aggregation degree A1 is larger than the threshold As (S207: YES), as shown in FIG. 7A, the control unit 3 obtains a concentration C1 of the target substance from the aggregation degree A1 based on the calibration curve T1 (S208), and terminates the measurement (S209). Then, the control unit 3 displays the obtained concentration C1 on the display input unit 2 (S210). In this manner, the step of measuring a specimen is terminated.

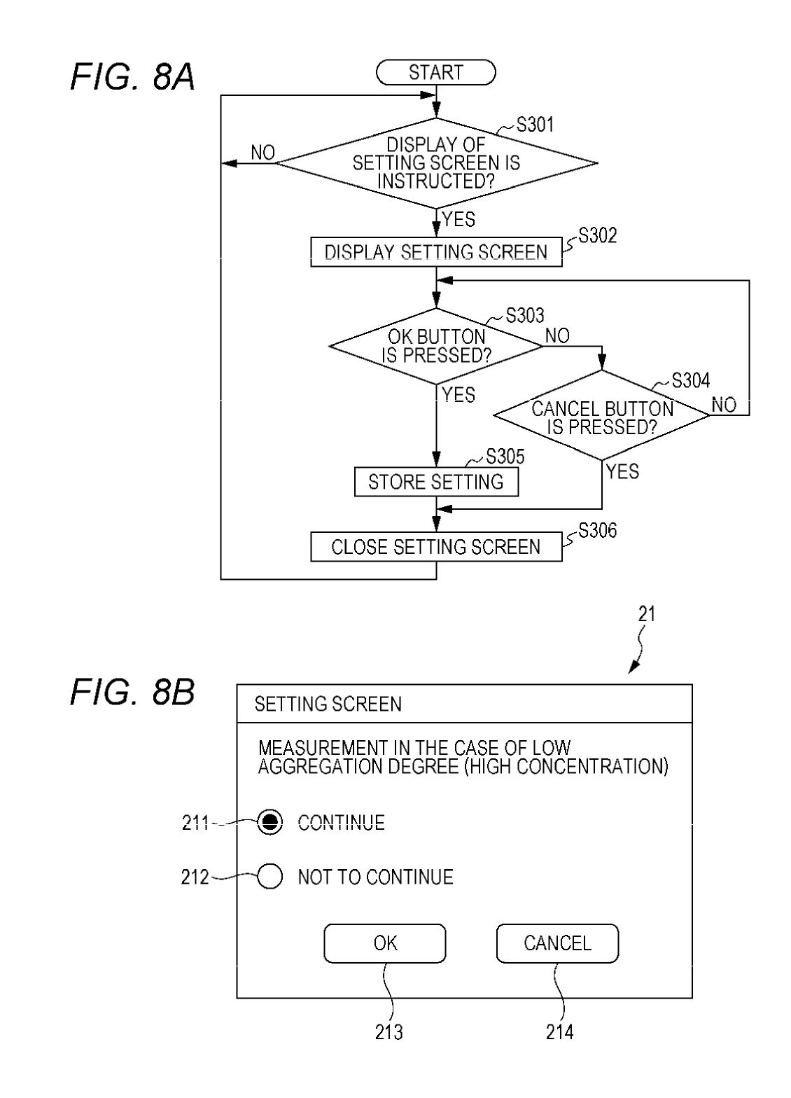

On the other hand, when the aggregation degree A1 is less than or equal to the threshold As (S207: NO), the control unit 3 determines whether the biological sample analyzer 1 continues the measurement based on the set contents stored in the memory unit 31 (S211). The set contents are previously set by a user via a setting screen 21. The step of storing the set contents and the setting screen 21 will be described later with reference to FIGS. 8A and 8B.

When it is set so that the biological sample analyzer 1 does not continue the measurement (S211: NO), steps S208 to S210 described above are performed. On the other hand, when it is set so that the biological sample analyzer 1 continues the measurement (S211: YES), the control unit 3 waits the step until the reaction time reaches t2 (S212). When the reaction time reaches t2 (S212: YES), the control unit 3 feeds only a predetermined amount of the measurement sample in the cuvette to the measurement unit 4, executes the measurement by the measurement unit 4, and calculates the aggregation degree A2 of the measurement sample as shown in FIG. 7C (S213).

Subsequently, the control unit 3 obtains the concentration C2 from the aggregation degree A2 based on the calibration curve T2 as shown in FIG. 7C (S214), and terminates the measurement (S215). Then the control unit 3 displays the obtained concentration C2 on the display input unit 2 (S216). In this manner, the step of measuring a specimen is terminated.

Thus, the slope (gradient) of the calibration curve T2 corresponding to the aggregation degree A2 is larger than the slope (gradient) of the calibration curve T1 corresponding to the aggregation degree A1. Thus, as shown in FIG. 7B, the concentration C2 obtained from the aggregation degree A2 based on the calibration curve T2 has higher accuracy than that of the concentration C1 obtained from the aggregation degree A1 based on the calibration curve T1.

FIG. 8A is a flow chart showing the step of setting executed by the control unit 3 and FIG. 8B is a view showing the setting screen 21 which is displayed on the display input unit 2.

With reference to FIG. 8A, when the user instructs the display of the setting screen 21 via the display input unit 2 (S301: YES), the control unit 3 displays the setting screen 21 on the display input unit 2 (S302). As shown in FIG. 8B, the setting screen 21 includes radio buttons 211 and 212, an OK button 213, and a cancel button 214.

When the user pushes the OK button 213 (S303: YES), the control unit 3 stores the set contents in the memory unit 31 depending on the radio button selected from the radio buttons 211 and 212 (S305). That is, when the OK button 213 is pushed in a state where the radio button 211 (continue) is selected, it is set that the biological sample analyzer 1 performs the measurement not using the calibration curve T1 but using the calibration curve T2 (executes S212 to S216) in the case where the aggregation degree A1 calculated in S206 of FIG. 6 is small. On the other hand, when the OK button 213 is pushed in a state where the radio button 212 (not continue) is selected, it is set that the biological sample analyzer 1 performs the measurement using the calibration curve T1 (executes S208 to S210) even in the case where the aggregation degree A1 calculated in S206 of FIG. 6 is small. When the set contents are stored (S305), the control unit 3 closes the setting screen 21 (S306).

When the user pushes the cancel button 214 (S304: YES), the control unit 3 discards the state of the radio buttons 211 and 212 and closes the setting screen 21 (S306). The set contents set by the user via the setting screen 21 are read from the memory unit 31 by the control unit 3 in S211 of FIG. 6, and used when determining whether the measurement is continued in S211.

Subsequently, in the case where a plurality of calibration curves are created, the range of the aggregation degree suitable for using the calibration curves (the range of concentration) is examined. The result will be described.

FIG. 9A is a view showing calibration curves T11 to T15 as for five reaction times according to the step of creating calibration curves of FIG. 5A which are actually created by the inventors of this application by using the standard samples (1) to (10), the reaction buffer solution, and the competitive reagent. In this regard, the calibration curves T11 to T15 are created using PAMIA-40i, manufactured by Sysmex Corporation.

In FIG. 9A, a horizontal axis shows the concentration of thyroxine (T4) and a vertical axis shows the aggregation degree. In FIG. 5A, calibration curves T1 and T2 in two reaction times t1 and t2 (320 seconds, 1370 seconds) are created. Here, calibration curves T11 to T15 in five reaction times (170 seconds, 320 seconds, 620 seconds, 870 seconds, and 1370 seconds) are created. In this regard, the calibration curves T12 and T15 correspond to the calibration curves T1 and T2 created in the step of creating calibration curves. The point that the curves are plotted from the standard sample (1) having a thyroxine concentration of 0 μg/dL is not illustrated for convenience.

Table 2 below shows five coefficients of variation (CV) corresponding to five aggregation degrees calculated when determining points on the calibration curves T11 to T15 of FIG. 9A. The following table shows only the standard sample (4) to (9).

TABLE 2

| Reaction | | | | | | |
|---|---|---|---|---|---|---|
| time | Thyroxine concentration (μg/dL) | | | | | |
| (sec.) | 1 | 2.5 | 5 | 10 | 25 | 50 |
| 170 | 4.08% | 8.96% | 5.64% | 3.87% | 12.73% | 35.35% |
| 320 | 13.36% | 8.01% | 7.57% | 3.26% | 15.33% | 31.45% |
| 620 | 16.01% | 8.65% | 6.52% | 2.48% | 3.81% | 13.67% |
| 870 | 24.98% | 8.11% | 5.21% | 5.09% | 3.67% | 10.52% |
| 1370 | 36.54% | 10.49% | 5.54% | 3.91% | 4.81% | 7.26% |

In this case, the coefficient of variation (CV) means variations in five concentrations c taken from five aggregation degrees v obtained at the time of creating of the calibration curve (for example, v1 and v2 obtained in S106 and S108 of FIG. 5) based on the obtained calibration curve as shown in FIG. 9B. When the coefficient of variation (CV) is small, there is a high possibility that variations in the concentration to be obtained are hardly caused.

According to Table 2 above, the case where the coefficient of variation (CV) becomes 10% or less is as follows: In the case where the reaction time is 170 seconds (the calibration curve T11), the concentration is from 1 to 10 μg/dL. In the case where the reaction time is 320 seconds (the calibration curve T12), the concentration is from 2.5 to 10 μg/dL. In the case where the reaction time is 620 seconds (the calibration curve T13), the concentration is from 2.5 to 25 μg/dL. In the case where the reaction time is 870 seconds (the calibration curve T14), the concentration is from 2.5 to 25 μg/dL. In the case where the reaction time is 1370 seconds (the calibration curve T15), the concentration is from 5 to 50 μg/dL. That is, the reaction time capable of calculating the concentration when the coefficient of variation (CV) is 10% or less is shorter as the concentration is low, and is longer as the concentration is high.

In this embodiment, in the case where a plurality of calibration curves are created, the range of the aggregation degree suitable for using the calibration curves (the range of concentration) is set based on the range in which the coefficient of variation (CV) is 10% or less. For example, as shown in FIG. 9C, taking into consideration the case of using the two calibration curves T12 and T15, a concentration range R1 suitable for using the calibration curve T12 is from 2.5 to 10 μg/dL, and a concentration range R2 suitable for using the calibration curve T15 is from 5 to 50 μg/dL. In this case, if the concentration obtained from the aggregation degree when the reaction time is 320 seconds in the specimen measurement based on the calibration curve T12 is included in the range R1, the accuracy of the concentration to be obtained is considered to be high. On the other hand, if the concentration obtained from the aggregation degree when the reaction time is 320 seconds in the specimen measurement based on the calibration curve T12 is larger than the range R1, the accuracy of the concentration to be obtained is considered to be low.

Then, for example, the threshold Cs related to the concentration is set within a range R3 in which the ranges R1 and R2 are overlapped. If the concentration obtained based on the calibration curve T12 is less than or equal to Cs, the concentration is obtained based on the calibration curve T12. If the concentration obtained based on the calibration curve T12 is larger than Cs, the concentration is obtained using not the calibration curve T12, but the calibration curve T15. In S113 of FIG. 5A, the threshold Cs of the concentration is thus calculated and the threshold As related to the aggregation degree corresponding to the threshold Cs is calculated based on the calibration curve T1.

As described above, according to this embodiment, the calibration curves T1 and T2 can be created by changing the reaction time from the same standard samples (1) to (10). Accordingly, the amounts of the standard sample, the reaction buffer solution, and the competitive reagent, which are necessary for creating the calibration curves, can be reduced, and the operation of the user which is necessary for creating the calibration curves, can be reduced.

According to this embodiment, the aggregation degree A1 obtained when the reaction time is t1 in the specimen measurement is larger than the threshold As, the concentration C1 is obtained using the calibration curve T1. On the other hand, if the aggregation degree A1 is less than or equal to the threshold As, the concentration C2 is obtained using the calibration curve T2 in the case of continuing the measurement. Thus, the calibration curves T1 and T2 created previously can be used properly so that the accuracy of the concentration to be obtained becomes high.

According to this embodiment, the user can set whether the biological sample analyzer 1 continues the measurement in the case of low aggregation degree (high concentration) via the setting screen 21 shown in FIG. 8B. Thus, the user can choose between obtaining high-precision measurement results and quickly obtaining measurement results depending on the measurement accuracy to be determined in the range of the low aggregation degree (high concentration). That is, as shown in FIG. 7B, in the case where the aggregation degree A1 obtained in the specimen measurement is less than or equal to the threshold As, it is usually possible to obtain high-precision measurement results (concentrations) based on the calibration curve T2 rather than the calibration curve T1. However, in order to obtain the measurement results based on the calibration curve T2, it is necessary to wait for the reaction of the measurement sample to progress until the reaction time t2 is passed. On the other hand, it is sufficient in some cases if the user can grasp most of the concentration of the target substance contained in the measurement sample. In such a case, desirably, the concentration of the target substance is presented to the user based on the calibration curve T1 at the timing of passing the reaction time t1 without waiting the reaction time t2. According to this embodiment, when the user previously sets not to continue the measurement based on the calibration curve T2, the accuracy is slightly reduced, but the measurement result based on the calibration curve T1 is presented to the user more rapidly.

The embodiments of the present invention have been described above. However, the embodiments of the present invention are not limited thereto.

For example, in the above embodiments, the target substance has been described as thyroxine (T4) which is a type of thyroid hormone. However, it is not limited thereto, and it may be other antigens. Further, the target substance may be not only the antigens but also antibodies.

In the above embodiments, polystyrene latex particles are used when preparing the competitive reagent. However, the particles are not particularly limited as long as they are particles used for immunoassay. Examples thereof include magnetic particles, polymer particles, metal oxide particles, glass particles, erythrocytes, and gelatin particles. As the polymer particles, latex particles are preferred.

Although the latex agglutination is used in the above embodiment, the ELISA method may be used.

In the above embodiments, the aggregation degree is calculated based on the forward scattered light signal obtained by the measurement unit 4 based on flow cytometry, and the concentration of the target substance is obtained based on the aggregation degree. However, the present invention is not limited thereto. The concentration of the target substance may be obtained based on the turbidity of the measurement sample obtained by a light source and a photodetector in the measurement unit 4. Further, other information may be used as long as the aggregation degree and the concentration of the target substance can be found.

In the above embodiments, two calibration curves T1 and T2 are created in the step of creating calibration curves of FIG. 5A. In the step of measuring a specimen of FIG. 6, either the calibration curve T1 or the calibration curve T2 is used based on one threshold As. However, the present invention is not limited thereto. In the step of creating calibration curves, three or more calibration curves may be created. In the step of measuring a specimen, based on a plurality of the thresholds As, any one of the calibration curves may be appropriately used.

In the above embodiments, two calibration curves T1 and T2 are created and then the step of measuring a specimen of FIG. 6 is performed in the step of creating calibration curves of FIG. 5A. However, the present invention is not limited thereto. It may be configured that, after obtaining the aggregation degrees A1 and A2 in the step of measuring a specimen of FIG. 6, in the step of creating calibration curves of FIG. 5A, two calibration curves T1 and T2 are created and the threshold As is calculated, and the concentration C1 or the concentration C2 is obtained based on the comparison results of the aggregation degree A1 and the threshold As.

Further, it is not necessary to always perform the step of creating calibration curves of FIG. 5A in the above embodiments whenever the step of measuring a specimen of FIG. 6 is performed. For example, when the reaction buffer solution and the competitive reagent which are used in the measuring step are changed, the step of creating calibration curves may be performed using the reaction buffer solution and the competitive reagent to be changed. That is, in the case where the reaction buffer solution and the competitive reagent which are used in the measuring step are the same as those used in the measuring step, the measuring step can be executed using the calibration curves T1 and T2 previously created in the step of creating calibration curves and the calculated threshold As.

In the above embodiments, when the aggregation degree A1 is less than or equal to the threshold As in the step of measuring a specimen of FIG. 6, the concentration C2 is obtained using the calibration curve T2 in the case of continuing the measurement. However, the present invention is not limited thereto. The concentrations C1 and C2 are obtained using the calibration curves T1 and T2, and then the display of the concentrations C1 and C2 may be selected based on the comparison results of the aggregation degree A1 and the threshold As. In this case, it suffices that, in the case where the aggregation degree A1 is larger than the threshold As, the concentration C1 is displayed, in the case where the aggregation degree A1 is less than or equal to the threshold As, the concentration C2 is displayed.

In the above embodiments, it is determined which the calibration curves T1 and T2 are used in order to obtain the concentration based on the information about whether the aggregation degree A1 obtained in S206 of FIG. 6 is larger than the threshold As. However, it may be determined based on the information about whether the concentration C1 corresponding to the aggregation degree A1 is smaller than the threshold Cs related to the concentration.

FIG. 10 is a flow chart showing the step of measuring a specimen in this case. The flow chart of FIG. 10 is a flow chart in which S207 and S208 are deleted in the flow chart of FIG. 6 and S217 and S218 are added. In this case, in S113 of FIG. 5A, the threshold Cs related to the concentration is previously calculated in place of the threshold As related to the aggregation degree. The threshold Cs is the concentration corresponding to the threshold As related to the calibration curve T1.

When the aggregation degree A1 is calculated with reference to FIG. 10 (S206), the control unit 3 obtains the concentration C1 from the aggregation degree A1 based on the calibration curve T1 (S217). In the case where the concentration C1 obtained in S217 is smaller than the threshold Cs (S218: YES), the control unit 3 moves the step to S209. On the other hand, in the case where the concentration C1 obtained in S217 is more than or equal to the threshold Cs (S218: NO), the control unit 3 moves the step to S211. In this case, similarly to the embodiment, the concentration of the target substance can be obtained with high accuracy based on the calibration curves T1 and T2.

In the above embodiments, when it is not set so that the biological sample analyzer 1 continues the measurement in S211 of FIG. 6 (S211: NO), steps S208 to S210 are performed. However, the present invention is not limited thereto. It may be configured that the information showing that the concentration is larger than the threshold Cs is displayed.

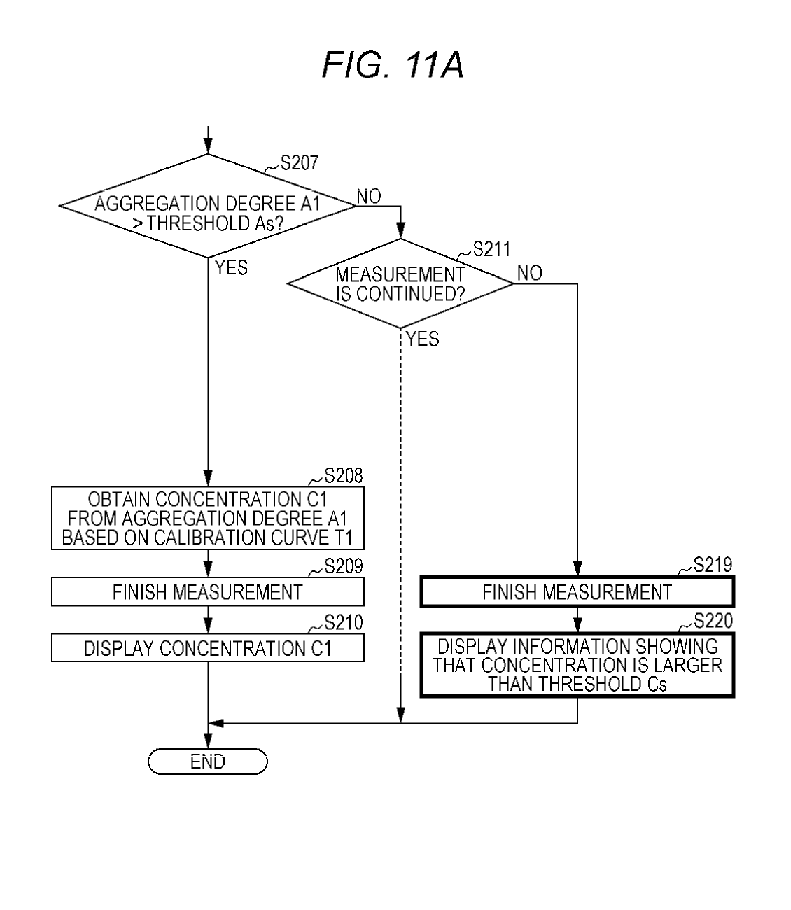

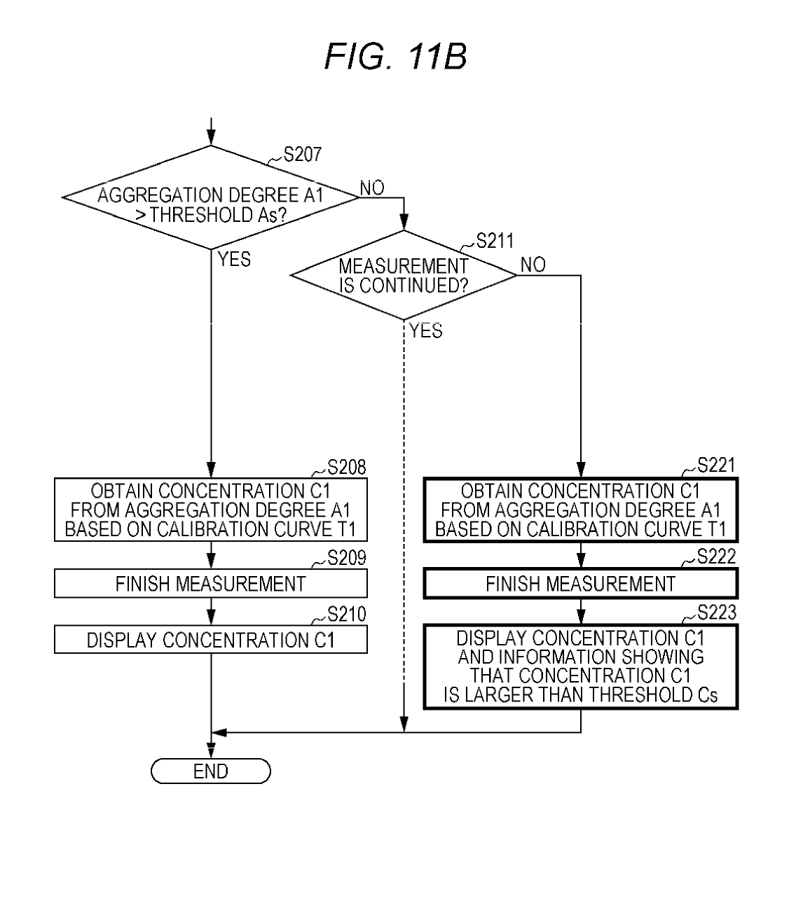

FIG. 11A is a flow chart showing the step of measuring a specimen in this case. The flow chart of FIG. 11A is a flow chart in which S219 and S220 are added in the flow chart of FIG. 6. For convenience, S201 to S206 and S212 to S216 are not illustrated.

With reference to FIG. 11A, when it is not set such that the biological sample analyzer 1 continues the measurement (S211: NO), the control unit 3 terminates the measurement (S219), and displays the information showing that the concentration is larger than the threshold Cs on the display input unit 2 (S220). Thus, the user can quickly obtain the information that the concentration of the target substance contained in the measurement sample exceeds the threshold. That is, in the case where the aggregation degree A1 obtained in the specimen measurement is less than or equal to the threshold As, it is necessary to wait for the reaction of the measurement sample to progress until the reaction time t2 is passed in order to obtain the measurement results based on the calibration curve T2. On the other hand, it is sufficient in some cases if the user can grasp whether the concentration of the target substance contained in the measurement sample exceeds the threshold Cs. In such a case, desirably, the information that the concentration of the target substance exceeds the threshold Cs or not is presented to the user based on the calibration curve T1 at the timing of passing the reaction time t1 without waiting the reaction time t2. According to the above configuration, when the user previously sets not to continue the measurement based on the calibration curve T2, the information that the concentration of the target substance exceeds the threshold Cs or not is presented to the user more rapidly.

In the case where the biological sample analyzer 1 is not set to continue the measurement (S211: NO), it suffices that the control unit 3 obtains the concentration C1 from the aggregation degree A1 based on the calibration curve T1, terminates the measurement (S221, S222) as shown in FIG. 11B, and displays the obtained concentration C1 and the information showing that the concentration C1 is larger than the threshold Cs on the display input unit 2 (S223). According to the type of the target substance or the value of the concentration to be obtained, an error or a warning message may be displayed on the display input unit 2 in S220 of FIG. 11A and S223 of FIG. 11B.

In addition, the embodiments of the present invention can be appropriately modified in various ways within the scope of the technical spirit shown in the claims.

What is claimed is:

1. A method of competitive immunoassay, comprising:
creating a first calibration curve created for a first reaction time period and a second calibration curve created for a second reaction time period longer than the first reaction time period;
preparing a measurement sample containing a target substance in a specimen, a reactant, and a competitive substance;
obtaining a reference information related to the concentration of the target substance after the first reaction time period since the preparation of the measurement sample;
first outputting the concentration obtained based on the first calibration curve as a measurement result in the case where the reference information shows that the concentration of the target substance is less than a predetermined threshold; and
second outputting the concentration obtained based on the second calibration curve as the measurement result in the case where the reference information obtained in the obtaining is information showing that the concentration of the target substance is more than or equal to the threshold.

2. The method according to claim 1, wherein in the obtaining step, the aggregation degree of the competitive substance in the measurement sample is obtained as the reference information.

3. The method according to claim 2, wherein in the first outputting step, the concentration obtained based on the first calibration curve is output as the measurement result when the aggregation degree obtained in the obtaining step exceeds a predetermined aggregation degree.

4. The method according to claim 2, wherein in the second outputting step, the concentration obtained based on the second calibration curve is output as the measurement result when the aggregation degree obtained in the obtaining step is less than or equal to the predetermined aggregation degree.

5. The method according to claim 2, wherein in the obtaining step, optical information of the measurement sample after the first reaction time period is obtained and the aggregation degree of the competitive substance is obtained based on the optical information.

6. The method according to claim 1, wherein in the obtaining step, the concentration of the target substance in the measurement sample is obtained as the reference information.

7. The method according to claim 6, wherein in the first outputting step, the concentration obtained based on the first calibration curve is output as the measurement result when the concentration obtained in the obtaining is less than a predetermined concentration.

8. The method according to claim 6, wherein in the second outputting step, the concentration obtained based on the second calibration curve is obtained as the measurement result when the concentration obtained in the obtaining is more than or equal to the predetermined concentration.

9. The method according to claim 6, wherein in the obtaining step, optical information of the measurement sample after the first reaction time period is obtained and the concentration of the target substance based on the optical information and the first calibration curve is obtained.

10. The method according to claim 1, wherein the concentration obtained based on the first calibration curve is output as the measurement result in the first outputting step without performing the second outputting step in the case where the reference information shows that the concentration of the target substance is less than the threshold.

11. The method according to claim 1, further comprising
setting whether to continue the measurement in the case where the reference information shows that the concentration of the target substance is more than or equal to the threshold,
wherein, in the case where the setting to continue the measurement is not made in the setting, when the reference information shows that the concentration of the target substance is more than or equal to the threshold, the concentration obtained based on the first calibration curve is output as the measurement result in the first outputting step without performing the second outputting step.

12. The method according to claim 1, further comprising:
setting whether to continue the measurement in the case where the reference information shows that the concentration of the target substance is more than or equal to the threshold,
wherein, in the case where the setting to continue the measurement is not made in the setting, when the reference information shows that the concentration of the target substance is more than or equal to the threshold, the information showing that the concentration of the target substance is more than or equal to the threshold is output as the measurement result without performing the second outputting step.

13. An apparatus for competitive immunoassay, comprising:
a sample preparation unit for preparing a measurement sample containing a target substance in a specimen, a reactant, and a competitive substance;
a detection unit; and
a control unit;
wherein the control unit:
stores a first calibration curve created for a first reaction time period and a second calibration curve created for a second reaction time period longer than the first reaction time period;
obtains predetermined reference information related to the concentration of the target substance after the first reaction time period since the preparation of the measurement sample based on the output of the detection unit;
outputs the concentration obtained based on the first calibration curve as the measurement result in the case where the obtained reference information shows that the concentration of the target substance is less than a predetermined threshold; and
outputs the concentration obtained based on the second calibration curve as the measurement result in the case where the reference information shows that the concentration of the target substance is more than or equal to the threshold.

14. The apparatus according to claim 13, wherein the control unit:
obtains the aggregation degree of the competitive substance in the measurement sample as the reference information;
outputs the concentration obtained based on the first calibration curve as the measurement result in the case where the aggregation degree exceeds a predetermined aggregation degree; and
outputs the concentration obtained based on the second calibration curve as the measurement result in the case where the aggregation degree is less than or equal to the predetermined aggregation degree.

15. The apparatus according to claim 14, wherein the control unit obtains optical information of the measurement sample after the first reaction time period and obtains the aggregation degree of the competitive substance based on the optical information.

16. An apparatus for competitive immunoassay, comprising:
a sample preparation unit configured to prepare a measurement sample containing a target substance in a specimen, a reactant, and a competitive substance;
a detection unit; and
a control unit;
wherein the control unit:
stores a first calibration curve created for a first reaction time period and a second calibration curve created for a second reaction time period longer than the first reaction time period;
obtains the concentration of the target substance after the first reaction time period since the preparation of the measurement sample based on the output of the detection unit;
outputs the concentration obtained based on the first calibration curve as the measurement result in the case where the obtained concentration shows that the concentration of a target substance is less than a predetermined threshold; and
outputs the concentration obtained based on the second calibration curve as the measurement result in the case where the obtained concentration shows that the concentration of the target substance is more than or equal to the threshold.

17. The apparatus according to claim 16, wherein the control unit outputs the concentration obtained based on the first calibration curve as the measurement result in the case where the concentration is less than a predetermined concentration, and outputs the concentration obtained based on the second calibration curve as the measurement result in the case where the concentration is more than or equal to the predetermined concentration.

18. The apparatus according to claim 16, wherein the control unit obtains optical information of the measurement sample after the first reaction time period and obtains the concentration of the target substance based on the obtained optical information and the first calibration curve.

19. The apparatus according to claim 13, further comprising:
a setting unit configured to set whether to continue the measurement in the case where the reference information shows that the concentration of the target substance is more than or equal to the threshold,
wherein, in the case where the setting to continue the measurement is not made by the setting unit, when the reference information is information showing that the concentration of the target substance is more than or equal to the threshold, the control unit outputs the concentration obtained based on the first calibration curve as the measurement result without measuring the concentration based on the second calibration curve.

20. The apparatus according to claim 13, further comprising:
a setting unit configured to set whether to continue the measurement in the case where the reference information shows that the concentration of the target substance is more than or equal to the threshold,
wherein, in the case where the setting to continue the measurement is not made by the setting unit, when the reference information shows that the concentration of the target substance is more than or equal to the threshold, the control unit outputs the information showing that the concentration of the target substance is more than or equal to the threshold as the measurement result without measuring the concentration based on the second calibration curve.
